(19) United States
(12) Patent Application Publication
(10) Pub. No.: US 2026/0158180 A1
(43) Pub. Date: Jun. 11, 2026

(54) ALBUMIN CONJUGATES AND PROCESS FOR PREPARATION THEREOF

(71) Applicant: Jubilant Draximage Inc., Kirkland (CA)

(72) Inventors: Indranil Nandi, Yardley, PA (US); Vijayaraj Kuniyil Kulangara, Kirkland (CA); Miriam Ruiz Pena, Kirkland (CA); Aida Mary Abreu Diaz, Kirkland (CA); Arundhati Bhattacharyya, Greater Noida (IN); Dinesh Kumar Sarwal, Greater Noida (IN)

(73) Assignee: Jubilant Draximage Inc., Kirkland (CA)

(21) Appl. No.: 19/538,692

(22) Filed: Feb. 12, 2026

Related U.S. Application Data
(63) Continuation of application No. 18/940,433, filed on Nov. 7, 2024.
(60) Provisional application No. 63/597,218, filed on Nov. 8, 2023.

(51) Int. Cl. A61K 51/12; A61B 6/40; A61J 1/06; A61K 9/00; A61K 9/19; A61K 31/175; A61K 31/198; A61K 31/282; A61K 31/337; A61K 31/475; A61K 31/495; A61K 31/573; A61K 31/704; A61K 31/7068; A61K 33/243; A61K 38/38; A61K 51/08

(52) U.S. Cl. CPC A61K 51/1241; A61K 9/0019; A61K 9/19; A61K 31/175; A61K 31/198; A61K 31/282; A61K 31/337; A61K 31/475; A61K 31/495; A61K 31/573; A61K 31/704; A61K 31/7068; A61K 33/243; A61K 38/385; A61K 51/081; A61K 51/088; A61B 6/4057; A61J 1/065; A61K 2121/00; A61K 2123/00

(57) ABSTRACT

The embodiments of the present invention disclose a conjugate compound of formula X-J-MAA, wherein X is a diagnostic or therapeutic radioisotope; J is an optional chelating agent; and MAA is macroaggregated albumin particles. The invention also discloses sterile and stable ready to use or reconstituted radiopharmaceutical composition of X-J-MAA and manufacturing process thereof, preferably the invention relates to radiopharmaceutical compositions of ^{99m}Tc. The prepared radiopharmaceutical compositions exhibit desirable technical attributes like stability, radiochemical purity, less impurities, pH, better bio-distribution and desirable average particle size for further administration to patients for therapeutic and diagnostic use.

### REFERENCE TO RELATED APPLICATIONS

This application is a continuation of application Ser. No. 18/940,433, filed on Nov. 7, 2024 and claims the benefit of U.S. Provisional Application No. 63/597,218, filed on Nov. 8, 2023, which is incorporated herein by reference.

### FIELD OF THE INVENTION

The present invention relates to radiopharmaceutical compositions of macroaggregated, microaggregated and nanoaggregated albumin for injection, a kit comprising the same and a process of manufacturing of the same. The radiopharmaceutical compositions can be used for diagnostic and/or therapeutic purposes.

### BACKGROUND OF THE INVENTION

Technetium ^{99m}Tc albumin aggregated (^{99m}Tc-MAA) is an injectable radiopharmaceutical formulation used in nuclear medicine. The formulation consists of a sterile aqueous suspension of Technetium-^{99m} (^{99m}Tc) labeled to human albumin aggregate particles. Reconstituted formulations are commonly used for lung perfusion diagnostic scanning. They are also used to visualise a peritoneovenous shunt and for isotope venography. Further, the lyophilized MAA kits available in the US market are multi-dose vials containing about 2 mg of macroaggregated albumin particles, about 0.05 mg of stannous chloride (as a ^{99m}Tc reducing agent), and about 5 mg of free human serum albumin. A lyophilized single dose Albumin Aggregated kit, radiolabeled with a radionuclide will offer application advantages over the currently available multiple dose products.

U.S. Pat. No. 6,730,286, discloses a process for preparation of technetium-^{99m} labelled macroaggregated albumin formulation. The process disclosed in the '286 patent requires purification of commercially available human serum albumin solution through ultrafiltration prior to proceeding with the aggregating step and requires passing the macroaggregates through a sizing screen after the macroaggregation step. Further, the process involving formation of a tin conjugate is a lengthy process involving heating at high temperature. The process suffers disadvantages in terms of requiring complex processes like ultrafiltration, size screening and long process time that makes the process complex, time consuming and economically inefficient for a large-scale application.

Importantly, macroaggregated particles of human serum albumin (known as macroaggregated albumin (MAA)) are expected to offer better radiopharmaceutical conjugates with other radioisotopes for diagnosis and therapeutic use. Some research publications have disclosed the application of MAA particles with other radioisotopes: a) MAA particles were studied for PET imaging of lung perfusion after radiolabeling with Gallium-68 (Ga-68) (Applied Radiation and Isotopes, 2008, 66 (12), 1910); b) MAA particles radiolabeled with Bismuth-212 (Bi-212) was evaluated for therapy of breast cancer (Frontiers in Chemistry, 2023, 11, 1204872); and c) MAA complexes with Eu(III) and Tb(III) were studied for magnetic resonance, positron emission and fluorescence imaging (Journal of the Turkish Chemical Society Section A: Chemistry, 2021, 8 (1), 209).

Macroaggregates of large size cause undesirable blockage of healthy lung and capillaries of other organs. Conversely, when macroaggregated particles are smaller than 10 microns in diameter, they pass through the walls of the capillaries directly into the liver and other parts of the body, causing undesirable shadows and distortion of the quality of the scintigraphic images. As per the United States Pharmacopoeia (USP) monograph for MAA, there should not be any particles larger than 150 μm and less than 10% should be below 10 μm. Similarly, the particle density or the number of particles in a unit dose is one of the most important characteristics for the effective and safe clinical performance of the radiolabeled macroaggregated albumin particles.

Therefore, the most desirable aspect of a process for preparation of macroaggregated or nanoaggregated albumin particles is its ability to provide consistent particle size distribution and particle density of MAA. However, macroaggregated albumin available from prior art known processes have variation in particle size distribution and particle density. It is reported that variation is observed in particle size distribution between different manufacturers, and even lot-to-lot variation between different batches of the same manufacturer (J Am Pharm Assoc (Wash). 2000, 40 (1), 46). This variability in particle size affects the quality, accuracy and reproducibility of the MAA radiolabeled products.

Radiolabeled nanoaggregates of Human Serum Albumin (HSA) are widely used in nuclear medicine for detection of the sentinel lymph node in many kinds of cancer by lymphoscintigraphy. Albumin nanoparticles of size range 30 nm to 100 nm are ideally suitable for localization in sentinel lymph nodes after interstitial injection. However, variability in particle size distribution and radioactivity distribution between products of different manufacturer has been reported (J. Label Compd. Radiopharm 2015, 58, 376).

There is an unmet need for a radiopharmaceutical drug product comprising macro/micro/nanoaggregated albumin with a specific desired particle size, uniform number of radiolabeled particles per dose, and chemical and radiochemical stability. These characteristics of the aggregated albumin allow their use as a regulatory and quality compliant drug product for diagnostic and/or therapeutic purposes. Consequently, there is a need for sterile injectable compositions of macro/micro/nanoaggregated albumin with desired technical attributes of particle size, particle density, pH and stability, which can be radiolabeled with a suitable diagnostic or therapeutic radioisotope for diagnostic and therapeutic purpose having desired radiochemical purity and radioactivity.

### SUMMARY OF THE INVENTION

The process for preparation of macroaggregated albumin products is based on a delicate heat, pH controlled denaturation and aggregation of Human Serum Albumin (HSA) under controlled stirring speeds. The present inventors have identified that desirable pharmaceutical manufacturing batches having high lot to lot uniformity in particle size distribution and particle density can be achieved using a robust, simple and time saving process without requirement of: a) undesirable ultrafiltration of starting material and b) extra separation and sieving steps by using a carefully designed manufacturing process with effective control of certain critical process parameters by using an automated reactor wherein process parameters are controlled by software for the preparation of the aggregated albumin product. Importantly, the most critical process parameters for the process of preparing MAA with the desired attributes are pH, rate of addition of reagents, stirring time, heating and cooling rates.

To achieve the desired technical and biological attributes, macroaggregated albumin is produced by controlling the physical aspects of the preparation process with an automated reactor with features including in-line pH monitoring using one or more sensitive pH probes; use of small peristatic pumps incorporated with the bioreactor to control the addition rates of the reagents; stirring with overhead stirrers with a specially designed impeller for desired stirring consistency; controlled heating and cooling using jacketed reaction vessels through which steam and chilled water is circulated as necessary. This keenly developed robust, simple and economic process resulted in desired particle density, particle size and mono-dispersity with precise control of stirring rate, pH and heating/cooling rate by software.

The present invention is provided in various aspects as outlined in the following:

The present invention relates to a pharmaceutical composition of macroaggregated albumin for injection and a pharmaceutical kit comprising the same. It also pertains to a process for manufacturing such a pharmaceutical composition and use thereof.

According to one aspect of the present invention, there is provided a conjugate compound of formula X-J-MAA, wherein X is a diagnostic or therapeutic radioisotope; J is an optional chelating agent; and MAA is macroaggregated albumin particles wherein the average particle size of the macroaggregated albumin particles is 25 μm to 35 μm, at least 95% of the macroaggregated albumin particles are between 10 μm to 70 μm, not more than 3% particles are below 10 μm and no particle is larger than 100 μm.

According to another aspect of the present invention, there is provided a sterile radiopharmaceutical composition, comprising: X-J-MAA, wherein X is a diagnostic or therapeutic radioisotope selected from the group consisting of copper-61, copper-64, copper-67, gallium-68, gallium-67, gallium-66, iodine-131, iodine-123, iodine-124, iodine-125, lutetium-177, yttrium-86, yttrium-90, technetium-^{99m}, indium-114, indium-111, scandium-47, scandium-44, scandium-43, terbium-149, zirconium-89, bismuth-213, bismuth-212, actinium-225, astatine-211, lead-212, lead-214, gold-198, gold-199, rhenium-188, rhenium-186, and holmium-166; J is an optional chelating agent; and MAA is macroaggregated albumin particles wherein the average particle size of the macroaggregated albumin particles is 25-35 μm, at least 95% of the macroaggregated albumin particles are between 10 μm to 70 μm, not more than 3% of the macroaggregated albumin particles are below 10 μm and none of the macroaggregated albumin particles is larger than 100 μm; and wherein the reconstituted and/or ready-to-use composition has not more than 5% soluble and dispersed radiochemical impurities; and wherein the reconstituted and/or ready-to-use composition has a pH of about 3.5 to about 8.5; and wherein the reconstituted and/or ready-to-use composition has bacterial endotoxin content less than 20 EU/vial; and wherein the reconstituted and/or ready-to-use composition has a radiochemical purity of at least 95% or more for at least 6 hours; and wherein the reconstituted and/or ready-to-use composition has a radioactivity of 0.185 MBq to 11100 MBq.

In yet another aspect of the present invention, there is provided a method of administering a sterile and stable radiopharmaceutical composition, the method comprising injecting a sterile radiopharmaceutical composition into a patient in need thereof, the radiopharmaceutical composition comprising X-J-MAA,
wherein X is a diagnostic or therapeutic radioisotope selected from the group consisting of copper-61, copper-64, copper-67, gallium-68, gallium-67, gallium-66, iodine-131, iodine-123, iodine-124, iodine-125, lutetium-177, yttrium-86, yttrium-90, technetium-^{99m}, indium-114, indium-111, scandium-47, scandium-44, scandium-43, terbium-149, zirconium-89, bismuth-213, bismuth-212, actinium-225, astatine-211, lead-212, lead-214, gold-198, gold-199, rhenium-188, rhenium-186, and holmium-166;
J is an optional chelating agent; and
MAA is macroaggregated albumin particles wherein the average particle size of the macroaggregated albumin particles is 20 to 35 μm, at least 95% of the macroaggregated albumin particles are between 10 μm to 70 μm, not more than 3% particles are below 10 μm and no particle is larger than 100 μm; and
wherein the reconstituted and/or ready-to-use composition has not more than 5% soluble and dispersed radiochemical impurities; and
wherein the reconstituted and/or ready-to-use composition has a pH of about 3.5 to about 8.5; and
wherein the reconstituted and/or ready-to-use composition has bacterial endotoxin content less than 20 EU/vial; and
wherein the reconstituted and/or ready-to-use composition has a radioactivity of 0.185 MBq to 11100 MBq; and
wherein the reconstituted and/or ready-to-use composition has a radiochemical purity of at least 95% or more for at least 6 hours after radiolabeling.

In one variation of the above composition, the average particle size of the macroaggregated albumin particles is 25 μm to 35 μm.

In another aspect, the present invention provides a sterile radiopharmaceutical composition of X-J-MAA prepared by a process, comprising the steps of:
i) preparing macroaggregated albumin (MAA) by:
   ia) mixing human serum albumin with a saline solution;
   ib) adjusting the pH to between 8.0 to 12.0 with stirring at a rate of 200 to 500 rpm at 60° C. to 80° C.;
   ic) cooling to 20 to 25° C. with stirring at rate of 200 to 400 rpm;
   id) adjusting the pH to 2.0 to 5.0 and sterile filtering the solution to obtain a filtrate;
   ie) adjusting the pH of the filtrate to 4.0 to 6.0;
   if) heating the solution at 50 to 80° C. at a stirring rate of 200 to 400 rpm and cooling to obtain macroaggregated albumin;
   ig) optionally, reacting the macroaggregated albumin with stannous chloride for 10 to 20 minutes at 65° C. to 80° C. to obtain stannated macroaggregated albumin (Sn-MAA) or conjugating macroaggregated albumin with a suitable chelating agent; and
   ih) optionally, introducing a chelating agent J;
ii) diluting the macroaggregated albumin or stannated macroaggregated albumin with water and adding excipients;
iii) optionally lyophilizing the reaction mixture of step ii); and
iv) radiolabeling with X;
wherein steps ia) to ig) are carried out as an automated continuous process; and
wherein the average particle size (e.g., diameter) of the macroaggregated albumin particles is 25 μm to 35 μm, at least 95% of the macroaggregated albumin particles are between 10 μm to 70 μm, not more than 3% particles are below 10 μm and no particle is larger than 100 μm; and
wherein the reconstituted and/or ready-to-use composition has not more than 5% soluble and dispersed radiochemical impurities; and
wherein the reconstituted and/or ready-to-use composition has a pH of 3.5 to 8.5; and
wherein the reconstituted and/or ready-to-use composition has bacterial endotoxin content less than 20 EU/vial; and
wherein the reconstituted and/or ready-to-use composition has a radioactivity of 0.185 MBq to 11100 MBq; and
wherein the reconstituted and/or ready-to-use composition has a radiochemical purity of at least 95% or more.

In another aspect, the present invention provides a sterile, non-pyrogenic, lyophilised pharmaceutical composition, comprising:
i) macroaggregated albumin;
ii) a reducing agent;
iii) human serum albumin; and
iv) an isotonicity agent;
wherein the macroaggregated albumin is stannous-incorporated macroaggregated albumin and wherein the average particle size of the macroaggregated albumin particles is 20-35 μm when measured by optical microscopy and/or automated microscopy and image analysis.

In another aspect, the present invention provides a reconstituted radiopharmaceutical composition, comprising:
(i) a first component comprising an aqueous solution of ^{99m}Tc pertechnetate and
(ii) a second component comprising a lyophilised pharmaceutical composition comprising:
   a) macroaggregated albumin,
   b) a reducing agent,
   c) human serum albumin, and
   d) an isotonicity agent,
wherein the macroaggregated albumin is stannated macroaggregated albumin having a particle density in the range of 300,000-700,000 particles in a unit dose and wherein the average particle size of the macroaggregated albumin particles is 25-35 μm, at least 95% of the macroaggregated albumin particles are between 10-70 μm, not more than 3% of the macroaggregated albumin particles are below 10 μm and none of the macroaggregated albumin particles are above 100 μm, when measured by optical microscopy and/or automated microscopy and image analysis; and wherein the composition comprises not more than 3% soluble and dispersed radiochemical impurities.

In yet another aspect, the present invention provides a sterile, non-pyrogenic, single dose, lyophilised pharmaceutical composition, comprising:
i) about 0.02 mg macroaggregated albumin;
ii) not less than 5 μg of stannous chloride;
iii) about 1.0 mg human serum albumin; and
iv) about 0.09-0.15 mg sodium chloride;
wherein the macroaggregated albumin is stannous incorporated macroaggregated albumin and wherein the average particle size of the macroaggregated albumin particles is 25-35 μm, 95% of the macroaggregated albumin particles are between 10-70 μm, less than 3% of the macroaggregated albumin particles are below 10 μm and none of the macroaggregated albumin particles are above 100 μm, when measured by optical microscopy and/or automated microscopy and image analysis;
wherein the composition comprises 300,000 to 800,000 macroaggregated albumin particles; and wherein the total tin content of the composition is not more than 13 μg;
wherein the pH of the composition when reconstituted with a 0.9% w/v sodium chloride solution is in the range of 5.0-7.0;
wherein the bacterial endotoxin content is less than 20 EU/vial; and
wherein the composition is contained in a vial comprising an oxygen content of less than 5 percent in the head space.

In another aspect of the present invention, there is provided a process for producing a lyophilised pharmaceutical composition of macroaggregated albumin for reconstitution and radiolabeling with ^{99m}Tc wherein the macroaggregated albumin is produced in a suitable reactor using an automated continuous process controlled by software.

In another aspect of the present invention, there is provided a lyophilized pharmaceutical composition of macroaggregated albumin, wherein upon reconstituting the composition with a ^{99m}Tc-pertechnate solution, the composition has a radiochemical purity of at least 99% for at least 12 hours at a temperature of 2° C. to 8° C.

In another aspect of the present invention, there is provided a lyophilized pharmaceutical composition of macroaggregated albumin, wherein the composition has a shelf-life of at least two years.

In another aspect of the present invention, there is provided a radiopharmaceutical composition of macroaggregated albumin, wherein the radiopharmaceutical composition is used for the diagnosis and/or treatment of ovarian cancer, breast cancer, liver cancer, prostate cancer, thyroid cancer, neuroendocrine tumors (NETs), pancreatic cancer, non-small cell lung cancer (NSCLC), and/or combinations thereof.

In another aspect of the present invention, there is provided a radiopharmaceutical composition of macroaggregated albumin, wherein the radiopharmaceutical composition is used for lung imaging, liver imaging, evaluation of pulmonary perfusion and peritoneovenous (LeVeen) shunt patency.

The details of one or more embodiments of the invention are set forth in the description below. Other features or advantages of the present invention will be apparent from the following figures and detailed description of several embodiments, and also from the appended claims.

### BRIEF DESCRIPTION OF THE DRAWINGS

The following figures form part of the present specification and are included to further illustrate certain aspects of the present disclosure, which can be better understood by reference to one or more of these drawings in combination with the detailed description of specific embodiments presented herein. It is to be understood that the data illustrated in the drawings in no way limit the scope of the disclosure. In the drawings:

FIG. 1: Illustrates the flow of process for the preparation of ^{99m}Technetium labelled MAA drug product.

FIG. 2: Illustrates the particle size distribution of the nanoaggregated albumin obtained by the process of present invention by following Example 1, Step 1, as determined by Dynamic Light Scattering method.

FIG. 3(a): Illustrates the uniformity of particle size distribution of three batches of Sn-MAA Bulk prepared by the process of present invention by following Example 1, Step 3, as determined by Malvern Morphologi G3. The almost superimposable graphs indicate minimal batch to batch variation.

FIG. 3(b) and FIG. 3(c): Illustrates the particle size distribution of two batches of Sn-MAA prepared by the process of present invention by following Example 1, Step 3 that shows the average particle size of both the batches are within the range 25 μm to 35 μm.

FIG. 4: Illustrates particle size distribution of Sn-MAA Bulk prepared by commercial process and process for the present invention. The graph corresponding to the reactor process is much narrower with sharp peak indicating monodispersed particles with low variation in size distribution.

FIG. 5: Illustrates the soluble and dispersed human serum albumin impurities content of three batches of Sn-MAA Bulk prepared by process of present invention by Following Example 1. The soluble and dispersed albumin content is less than 0.2% for all three batches.

FIG. 6: Illustrates the process flow of the preparation process of X-J-MAA.

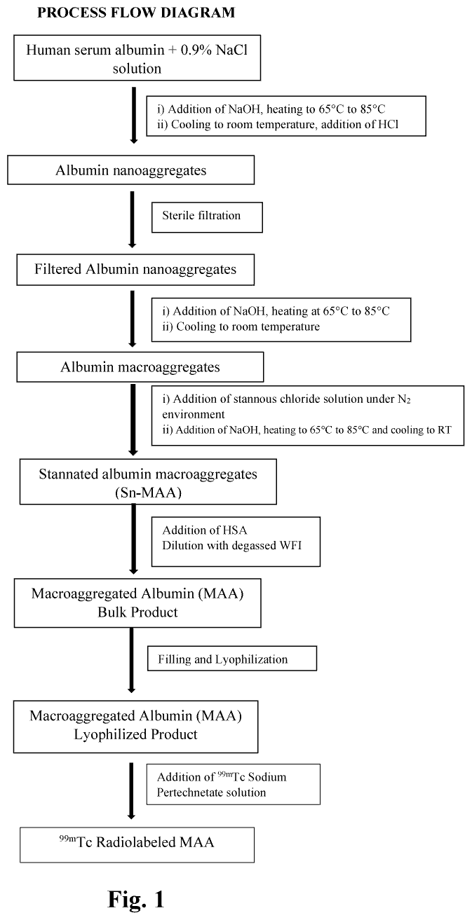

### DETAILED DESCRIPTION OF THE INVENTION

As used herein, the term “radionuclide or radioisotope” refers to an unstable form of a chemical element that releases radiation as it breaks down and becomes more stable. Radionuclides can occur in nature or can be generated in a laboratory. In medicine, they are used in the diagnosis and/or treatment of diseases.

As used herein, the term “dose” refers to the dose of a radionuclide required to perform imaging in a subject. The dose of a radionuclide to be administered into the subject ranges from 0.185 MBq to 11100 Mbq, wherein MBq is Megabecquerel.

The term “macroaggregated albumin” as used herein refers to conglomerates of denatured albumin with a particle size in the range of 5 μm to 150 μm.

The term “nanoaggregated albumin” as used herein refers to conglomerates of denatured albumin with a particle size in the range of 5 nm to 600 nm.

The term “stannated macroaggregated albumin” or “stannous incorporated macroaggregated albumin” as used herein refers to conglomerates of denatured albumin complexed with stannous (Sn²⁺) ion.

The term “human serum albumin” as used herein refers to serum albumin found in human blood which may be isolated from its natural source or prepared by recombinant DNA technology.

The term “isoelectric point” as used herein refers to the pH at which a molecule carries no net electrical charge or is electrically neutral. The isoelectric point for albumin ranges from 4.7 to 5.2.

The term “API” as used herein refers to the active pharmaceutical ingredient that enables radionuclide activity at desired site.

The term “X-J-MAA” as used herein refers to a radioconjugate comprising at least one radionuclide (X), which enables the imaging and/or the destruction of the cancer cells, and macroaggregated albumin (MAA) with effective particle density and particle size and optionally a chelating agent (J) enabling at least one radionuclide to be attached to macroaggregated albumin.

As used herein the term “radiochemical purity” refers to the proportion of the total radioactivity in the sample, which is present as the desired radiolabeled species. Radiochemical purity is important since it is the radiochemical form, which determines the bio-distribution of the radiopharmaceutical. The present invention includes the pharmaceutical composition, wherein the composition has a radiochemical purity of at least 95 percent, preferably 97 percent, most preferably 99 percent, most preferably 100 percent.

The term “single dose kit” as used herein, refers to a kit containing a single dose of the pharmaceutically active ingredient.

The term “multiple dose kit” or “multi-dose kit” as used herein, refers to a kit containing more than one dose of the pharmaceutically active ingredient.

The term “reactor” as used herein refers to a type of reaction vessel that is used for the production of various chemicals and biological conversions and can include chemical reactors, reaction vessels and bioreactors. The reactors of the present invention can be an automated reactor with software control equipped with sensitive pH probes for in-line pH monitoring; small peristatic pumps to control the addition rates of the reagents; overhead stirrers with specially designed impeller for desired stirring rate; jacketed reaction vessels with circulating steam and/or chilled water for controlled heating and cooling. The process parameters such as heating rate, cooling rate, heating set point, cooling set point, addition rate of reagents, pH, and stirring rate can be pre-programmed and can be precisely controlled with the software. The reactors of the present invention may comprise multiple interconnected reaction vessels of size ranging from 1 L to 50 L. The reactors of the present invention can be made of glass, ceramic, polymer or stainless steel materials.

The terms “lyophilization” or “lyophilisation” are interchangeable and as used herein, refer to a low temperature dehydration process that involves freezing the product and lowering pressure, thereby removing the ice by sublimation. The term lyophilisation as used herein is synonymous with freeze-drying. The lyophilisation process consists of three separate stages: freezing, primary drying (sublimation), and secondary drying (desorption).

The term “about” as used herein, refers to a measurable value such as a parameter, an amount, a temporal duration, and the like, and is meant to encompass variations of and from the specified value, in particular variations of ±10% or less, preferably ±5% or less, more preferably ±1% or less, and still more preferably ±0.1% or less of and from the specified value, such variations are appropriate to perform in the disclosed invention. It is to be understood that the value to which the modifier “about” refers is itself also specifically, and preferably, disclosed.

The term “reconstituted” as used herein, refers to radiopharmaceutical compositions prepared from non-radioactive kits by radiolabeling with radionuclide prior to use.

The term “ready to use” as used herein, refers to a liquid radiopharmaceutical injectable composition which can be directly injected into a human or an animal without the need of any prior preparation.

The term “SDV” as used herein, is an abbreviation of single dose vial.

The term “MDV” as used herein, is an abbreviation of multiple dose vial.

According to one embodiment of the present invention, there is provided aggregated protein-based particles with size ranging from 2 nm to 100 μm.

According to one embodiment of the present invention, there is provided aggregated protein-based particles with size ranging from 2 nm to 100 μm with narrow size distribution and polydispersity index less than 0.5, prepared using a continuous automated manufacturing process in an automated reactor wherein the protein is chosen from albumin, globulin, gelatin, whey protein, gliadin, legumin, elastin, zein, soy protein, and milk protein, ferritin, transferrin etc.

According to a preferred embodiment, the protein is albumin.

According to one embodiment of the present invention, there is provided macroaggregated albumin particles with size ranging from 10 μm to 100 μm wherein at least 95% particles are within the size range of 10 μm to 70 μm.

According to one embodiment of the present invention, there is provided nanoaggregated albumin particles with size ranging from 5 nm to 200 nm wherein the polydispersity index of the nanoaggregated particles is ≤0.4.

According to one embodiment of the present invention, there is provided a process for producing aggregated protein-based particles with size ranging from 2 nm to 100 μm with narrow particle size distribution and a polydispersity index less than 0.5, using an automated manufacturing process in a suitable reactor.

According to one embodiment of the present invention, there is provided macroaggregated albumin particles with size ranging from 10 μm to 100 μm, wherein at least at least 95% of the macroaggregated albumin particles are between 10 μm to 70 μm, not more than 3% particles are below 10 μm and no particle is larger than 100 μm.

According to one embodiment, the particle size distribution and particle density of the macroaggregated albumin particles can be measured by different methods such as optical microscopy, automated microscopy and image analysis, laser diffraction, dynamic light scattering, Scanning Electron Microscopy (SEM), centrifugation test and filtration test.

According to one embodiment, the particle size distribution and particle density of the macroaggregated albumin particles are measured by automated microscopy and image analysis.

According to one embodiment, the particle size distribution and particle density of the macroaggregated albumin particles are measured by optical microscopy using a hemocytometer.

According to one embodiment of the present invention, there is provided a conjugate compound of formula X-J-MAA,
wherein X is a diagnostic or therapeutic radioisotope;
J is an optional chelating agent; and
MAA is macroaggregated albumin particles wherein the average particle size of the macroaggregated albumin particles is 25 μm to 35 μm, at least 95% of the macroaggregated albumin particles are between 10 μm to 70 μm, not more than 3% particles are below 10 μm and no particle is larger than 100 μm.

According to another embodiment of the present invention, there is provided a sterile radiopharmaceutical composition, comprising:

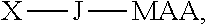

wherein X is a diagnostic or therapeutic radioisotope selected from the group consisting of copper-61, copper-64, copper-67, gallium-68, gallium-67, gallium-66, iodine-131, iodine-123, iodine-124, iodine-125, lutetium-177, yttrium-86, yttrium-90, technetium-^{99m}, indium-114, indium-111, scandium-47, scandium-44, scandium-43, terbium-149, zirconium-89, bismuth-213, bismuth-212, actinium-225, astatine-211, lead-212, lead-214, gold-198, gold-199, rhenium-188, rhenium-186, and holmium-166;
J is an optional chelating agent; and
MAA is macroaggregated albumin particles wherein the average particle size of the macroaggregated albumin particles is 25-35 μm, at least 95% of the macroaggregated albumin particles are between 10 μm to 70 μm, not more than 3% particles are below 10 μm and no particle is larger than 100 μm; and
wherein the reconstituted and/or ready-to-use composition has not more than 5% soluble and dispersed radiochemical impurities; and
wherein the reconstituted and/or ready-to-use composition has a pH of about 3.5 to about 8.5; and
wherein the reconstituted and/or ready-to-use composition has bacterial endotoxin content less than 20 EU/vial; and
wherein the reconstituted and/or ready-to-use composition has a radiochemical purity of at least 95% or more for at least 6 hours;
wherein the reconstituted and/or ready-to-use composition has a radioactivity of 0.185 MBq to 11100 MBq.

According to another embodiment of the present invention, there is provided a method of administering a sterile and stable radiopharmaceutical composition, the method comprising injecting a sterile radiopharmaceutical composition into a patient in need thereof, the radiopharmaceutical composition, comprising X-J-MAA,
wherein X is a diagnostic or therapeutic radioisotope selected from the group consisting of copper-61, copper-64, copper-67, gallium-68, gallium-67, gallium-66, iodine-131, iodine-123, iodine-124, iodine-125, lutetium-177, yttrium-86, yttrium-90, technetium-^{99m}, indium-114, indium-111, scandium-47, scandium-44, scandium-43, terbium-149, zirconium-89, bismuth-213, bismuth-212, actinium-225, astatine-211, lead-212, lead-214, gold-198, gold-199, rhenium-188, rhenium-186, and holmium-166;
J is an optional chelating agent; and
MAA is macroaggregated albumin particles wherein the average particle size of the macroaggregated albumin particles is 25-35 μm, at least 95% of the macroaggregated albumin particles are between 10 μm to 70 μm, not more than 3% particles are below 10 μm and no particle is larger than 100 μm; and
wherein the reconstituted and/or ready-to-use composition has not more than 5% soluble and dispersed radiochemical impurities; and
wherein the reconstituted and/or ready-to-use composition has a pH of about 3.5 to about 8.5; and
wherein the reconstituted and/or ready-to-use composition has bacterial endotoxin content less than 20 EU/vial; and
wherein the reconstituted and/or ready-to-use composition has a radioactivity of 0.185 MBq to 11100 MBq; and
wherein the reconstituted and/or ready-to-use composition has a radiochemical purity of at least 95% or more for at least 6 hours after radiolabeling.

In one embodiment, X is a diagnostic or therapeutic radioisotope selected from the group consisting of copper-61, copper-64, copper-67, gallium-68, gallium-67, gallium-66, iodine-131, iodine-123, iodine-124, iodine-125, lutetium-177, yttrium-86, yttrium-90, technetium-^{99m}, indium-114, indium-111, scandium-47, scandium-44, scandium-43, terbium-149, zirconium-89, bismuth-213, bismuth-212, actinium-225, astatine-211, lead-212, lead-214, gold-198, gold-199, rhenium-188, rhenium-186, and holmium-166.

In one embodiment, the chelating agent J is selected from group consisting of: 2,2′,2″,2″-(1,4,7,10-Tetraazacyclododecane-1,4,7,10-tetrayl)tetraacetic acid (DOTA), 2,2′,2″-(1,4,7-triazacyclononane-1,4,7-triyl)triacetic acid (NOTA), trientine hydrochloride (TETA), Diethylenetriamine pentaacetate (DTPA), 1,4-bis(carboxymethyl)-6-[bis(carboxymethyl)]amino-6-methylperhydro-1,4-diazepine) (AAZTA), Nitrilotriacetic acid (NTA), ethylenediaminetetraacetic acid (EDTA), 1,4,7,10-Tetraazacyclododecane-1,4,7-triacetic acid (DO3A), NOC, DOTAGA, hydroxybenzyl ethylenediamine (HBED), HBECC, 1,4,7-triazacyclononane, 1-glutaric acid-4,7-acetic acid (NODAGA), desferrioxamine (DFO), N,N′-bis(2,2-dimethyl-2-mercaptoethyl)ethylenediamine-N,N′-diacetic acid (6SS), 1-(4-carboxymethoxybenzyl)-N—N′-bis[(2-mercapto-2,2-dimethyl)ethyl]-1,2-ethylenediamine-N,N′-diacetic acid (B6SS), PLED (N,N′-dipyridoxylethylenediamine-N,N′-diacetic acid), TAME, YM103, 1,2-bis[[(6-carboxypyridin-2-yl)methyl]amino]ethane (H2dedpa), and combinations thereof.

In another embodiment, the macroaggregated albumin comprises not more than 3% soluble and dispersed human serum albumin impurities content when measured by automated microscopy and image analysis.

In another embodiment, the radiopharmaceutical composition of X-J-MAA may further comprises of one or more pharmaceutically acceptable excipients selected from:
a buffer selected from acetate, phosphate, formate, citrate, maleate, malate, carbonate, bicarbonate, HEPES, borate and combinations thereof;
a radioprotectant or radiochemical stabilizer selected from ascorbic acid, gentisic acid, p-amino benzoic acid (PABA), nicotinic acid, nicotinamide; benzyl alcohol, ethanol, cysteine, methionine and combinations thereof;
an isotonicity agent selected from sodium chloride, dextrose, mannitol, dextran;
a bulking agent selected from albumin, sodium chloride, dextrose, lactose, sucrose, mannitol, sorbitol, inositol, glycine and combinations thereof;
a metal inhibitor or transchelator selected from the group consisting of DTPA, EDTA, beta cyclodextrin, monosachharides, oligosaccharides, polysaccharides, sulphated sugars and combinations thereof;
and optionally, a reducing agent selected from stannous chloride, stannous fluoride, and combinations thereof.

In one embodiment the sterile radiopharmaceutical composition that is reconstituted and/or ready-to-use composition has a pH drift of less than 0.5.

In one embodiment, the reconstituted and/or ready-to-use composition has a pH drift of less than 0.2.

In one embodiment, the composition comprising X-J-MAA is a lyophilized composition.

In another embodiment, the composition comprising X-J-MAA is packaged in a clinical grade container selected from vials, syringes, ampoules, and pre-filled syringes.

In another embodiment, the sterile radiopharmaceutical composition comprising X-J-MAA is packaged in a radiation shielded pre-filled syringe.

In another embodiment, the vial is a sterile pyrogen-free glass vial of Type 1 glass with a fluorocoated bromobutyl rubber septum.

In another embodiment, the aforementioned rubber septum is sealed with a crimped aluminum capsule.

In another embodiment, the packaging for the sterile radiopharmaceutical composition may further comprise a lead shielded transport container, wherein the glass vial, syringe is kept in a lead shielded container during transportation.

In another embodiment, the lead shielded transport container complies with type A requirements (IAEA standards).

In one embodiment, the present invention provides a single dose kit comprising the pharmaceutical composition of X-J-MAA, wherein the number of macroaggregated particles in the kit is between 300,000 to 700,000.

In another embodiment, the present invention provides a multiple dose kit comprising the pharmaceutical composition of X-J-MAA, wherein the number of macroaggregated particles in the kit is between 3,000,000 to 7,000,000.

In one embodiment, the present invention provides a sterile radiopharmaceutical composition of X-J-MAA prepared by a process, comprising the steps of:
i) preparing macroaggregated albumin (MAA) by:
   ia) mixing human serum albumin with a saline solution;
   ib) adjusting the pH to between 8.0 to 12.0 with stirring at a rate of 200 to 500 rpm at 60° C. to 80° C.;
   ic) cooling to 20 to 25° C. with stirring at a rate of 200 to 400 rpm;
   id) adjusting the pH to 2.0 to 5.0 and sterile filtering the solution to obtain filtrate;
   ie) adjusting the pH of the filtrate to 4.0 to 6.0
   if) heating the solution at 50° C. to 80° C. at a stirring rate of 200 to 400 rpm and cooling to obtain macroaggregated albumin;
   ig) optionally, reacting macroaggregated albumin with stannous chloride for 10 to 20 minutes at 65° C. to 80° C. to obtain stannated macroaggregated albumin (Sn-MAA) or conjugating macroaggregated albumin with a suitable chelating agent; and
   ih) optionally, introducing a chelating agent J;
ii) diluting the macroaggregated albumin or stannated macroaggregated albumin with water and adding excipients;
iii) optionally lyophilizing the reaction mixture of step ii); and
iv) radiolabeling with X;
wherein steps ia) to ig) are carried out as automated continuous process; and
wherein the average particle size of the macroaggregated albumin particles is 20 μm to 35 μm, at least 95% of the macroaggregated albumin particles are between 10 μm to 70 μm, not more than 3% particles are below 10 μm and no particle is larger than 100 μm; and
wherein the reconstituted and/or ready-to-use composition has not more than 5% soluble and dispersed radiochemical impurities; and
wherein the reconstituted and/or ready-to-use composition has a pH of 3.5 to 8.5; and
wherein the reconstituted and/or ready-to-use composition has bacterial endotoxin content less than 20 EU/vial; and
wherein the reconstituted and/or ready-to-use composition has a radioactivity of 0.185 MBq to 11100 MBq; and
wherein the reconstituted and/or ready-to-use composition has a radiochemical purity of at least 95% or more.

In one embodiment, the above process does not comprise initial purification of the albumin used as the starting material; and, wherein the process does not comprise separation of the stannous incorporated macroaggregated albumin particles and passing the same through a sizing screen.

In one embodiment, in the above process the macroaggregated albumin after step i) has a pH drift of less than 0.4 over 24 hours, and lyophilized product after step iv) has a pH drift of less than 0.5 when stored for at least six months at room temperature and reconstituted with 0.9% saline and has no pH drift over 72 hours after reconstitution.

In another embodiment, the sterile radiopharmaceutical composition of X-J-MAA is used for the diagnosis and/or treatment of ovarian cancer, breast cancer, liver cancer, prostate cancer, thyroid cancer, neuroendocrine tumors (NETs), pancreatic cancer, non-small cell lung cancer (NSCLC), and/or combinations thereof.

In another embodiment, the sterile radiopharmaceutical composition of X-J-MAA is used for the detection of extrahepatic perfusion to non-target organs prior to radioembolization therapy of unresectable primary and metastatic liver cancer after intra-arterial administration into the hepatic artery.

In yet another embodiment, the sterile radiopharmaceutical composition of X-J-MAA is co-administered with one or more pharmaceutically acceptable therapeutic agents selected from the group consisting of Cabazitaxel, Docetaxel, Nab-paclitaxel, Paclitaxel; Vinblastine, Prednisone, Methylprednisolone, Dexamethasone, Daunorubicin, Epirubicin, Idarubicin, Valrubicin, Gemcitabine, Methotrexate, Pralatrexate, Azacitidine, 5-fluorouracil (5-FU), 6-mercaptopurine (6-MP), Capecitabine, Cladribine, Clofarabine, Cytarabine, Decitabine, Floxuridine, Fludarabine, Carmustine, Lomustine, Streptozocin, Cisplatin, Cyclophosphamide, Dacarbazine, Ifosfamide, Mechlorethamine, Melphalan, Oxaliplatin, Temozolomide, Thiotepa, Altretamine, Bendamustine, Busulfan, Carboplatin and combinations thereof for the treatment of ovarian cancer, breast cancer, liver cancer, prostate cancer, thyroid cancer, neuroendocrine tumors (NETs), pancreatic cancer, non-small cell lung cancer (NSCLC), and/or combinations thereof

In a preferred embodiment, the present invention provides a sterile, non-pyrogenic, lyophilised pharmaceutical composition, comprising:
i) macroaggregated albumin;
ii) a reducing agent;
iii) human serum albumin; and
iv) an isotonicity agent;
wherein the macroaggregated albumin is stannous incorporated macroaggregated albumin and wherein the average particle size of the macroaggregated albumin particles is 25 μm to 35 μm when measured by optical microscopy and/or automated microscopy and image analysis.

In another embodiment, at least 95% of the macroaggregated albumin particles are between 10-70 μm, less than 3% of the particles are below 10 μm and none of the particles are above 100 μm and additionally have not more than 3% soluble and dispersed human serum albumin impurities content, when measured by optical microscopy and/or automated microscopy and image analysis.

In another embodiment, the macroaggregated albumin particles have not more than 3% soluble and dispersed human serum albumin content, when measured by optical microscopy and/or automated microscopy and image analysis.

In another embodiment, the pH of the lyophilized pharmaceutical composition when reconstituted with a 0.9% w/v sodium chloride solution is 5.0 to 7.0.

In another embodiment, the bacterial endotoxin content of the lyophilized pharmaceutical composition is not more than 20 EU/vial.

In another embodiment, the lyophilized pharmaceutical composition is contained in a vial comprising an oxygen content of less than 5 percent in the head space.

In accordance with another embodiment, the reducing agent is selected from group consisting of stannous chloride, stannous fluoride, sodium dithionite, sodium bisulphite, formamidine sulphonic acid, sodium thiosulfate, stannous sulfide and combinations thereof.

In accordance with another embodiment, the isotonicity agent is selected from sodium chloride, dextrose and mannitol.

In a preferred embodiment, the isotonicity agent is sodium chloride.

According to one embodiment, the macroaggregated albumin is prepared by an automated manufacturing process in a suitable reactor.

In accordance with another embodiment, the lyophilised pharmaceutical composition comprises the macroaggregated albumin and the human serum albumin in a ratio of 1:5 to 1:25.

In one embodiment, the present invention provides a kit comprising the aforementioned lyophilized pharmaceutical composition, wherein the kit comprises a clinical grade container selected from vials, syringes, ampoules, and pre-filled syringes.

In accordance to one embodiment, the aforementioned kit comprises a glass vial of capacity 3 mL to 30 mL.

In one embodiment of the present invention, particle size and particle density of the lyophilized pharmaceutical composition is maintained at the initial value for at least 2 years of storage at room temperature.

In another embodiment of the present invention, the pH of the lyophilized pharmaceutical composition after reconstitution with a 0.9% sodium chloride solution does not show a drift of more than 0.5 for at least 2 years of storage at room temperature.

In accordance with one aspect of the present invention, the invention provides a reconstituted radiopharmaceutical composition, comprising:
(i) a first component comprising an aqueous solution of ^{99m}Tc pertechnetate and
(ii) a second component comprising a lyophilised pharmaceutical composition comprising:
   a) macroaggregated albumin,
   b) a reducing agent,
   c) human serum albumin, and
   d) an isotonicity agent,
wherein the macroaggregated albumin is stannated macroaggregated albumin having a particle density in the range of 300,000 to 700,000 particles in a unit dose and wherein the average particle size of the macroaggregated albumin particles is 25 μm to 35 μm, at least 95% of the macroaggregated albumin particles are between 10-70 μm, not more than 3% particles are below 10 μm and none are above 100 μm, when measured by optical microscopy and/or automated microscopy and image analysis; and wherein the composition comprises not more than 3% soluble and dispersed radiochemical impurities.

In one embodiment, the reconstituted radiopharmaceutical composition comprises radioactivity in the range of 20 to 500 megabecquerels (MBq).

In one embodiment, the present invention provides a process for the preparation of radiopharmaceutical composition, comprising: reconstituting the a lyophilized composition of i) macroaggregated albumin; ii) a reducing agent; iii) human serum albumin; and iv) an isotonicity agent, wherein the macroaggregated albumin is stannous incorporated macroaggregated albumin and wherein the average particle size of the macroaggregated albumin particles is 20-35 μm when measured by optical microscopy and/or automated microscopy and image analysis with (a) a sterile solution of ^{99m}Tc-pertechnetate or (b) first a biocompatible carrier followed by a sterile solution of ^{99m}Tc pertechnetate.

In one embodiment, the said reconstituted radiopharmaceutical composition has a radiochemical purity of at least 99% for at least 12 hours when stored at a temperature of 2° C. to 8° C.

In another embodiment, not more than 10% of the total radioactivity is observed in the supernatant liquid after centrifugation of the said reconstituted radiopharmaceutical composition at about 2000 rpm for 5 minutes to 10 minutes.

In another embodiment, not less than 85.0% of the radioactivity is found in the lungs, not more than 2.0% of the radioactivity is found in the liver, and not more than 2.0% of the radioactivity is found in the kidneys when formulation tested for bio-distribution in not less than two animals, wherein 0.074 MBq and 0.740 MBq of the said reconstituted radiopharmaceutical composition is injected into three suitable animals selected from mice and rats.

In one embodiment of the present invention, the reconstituted radiopharmaceutical composition is used for lung imaging, evaluation of pulmonary perfusion and peritoneovenous (LeVeen) shunt patency.

In another embodiment of the present invention, the reconstituted radiopharmaceutical composition is used for the diagnosis and/or treatment of ovarian cancer, breast cancer, liver cancer, prostate cancer, thyroid cancer, neuroendocrine tumors (NETs), pancreatic cancer, non-small cell lung cancer (NSCLC), and/or combinations thereof.

In another embodiment of the present invention, the reconstituted radiopharmaceutical composition is used for detection of extrahepatic perfusion to non-target organs prior to radioembolization therapy of unresectable primary and metastatic liver cancer after intra-arterial injection into the hepatic artery.

In one embodiment of the present invention, the volume of one dose of the reconstituted radiopharmaceutical composition is from about 0.1 mL to 3 mL.

In accordance with one embodiment, the present invention provides a process for preparing the aforementioned reconstituted radiopharmaceutical composition, comprising the steps of:
i) preparing stannous macroaggregated albumin (Sn-MAA) by
   ia) mixing human serum albumin with saline solution;
   ib) adjusting the pH between 8.0 to 12.0 with stirring at a rate of 200 to 500 rpm at a suitable temperature;
   ic) cooling to 20° C. to 25° C. with stirring at a rate of 200 to 400 rpm;
   id) adjusting the pH to 2.0 to 5.0 and sterile filtering the solution to obtain filtrate;
   ie) adjusting the pH of the filtrate to 4.0 to 6.0;
   if) heating the solution at 50° C. to 80° C. at a stirring rate of 200 to 400 rpm and cooling to obtain macroaggregated albumin; and
   ig) reacting the macroaggregated albumin with stannous chloride to obtain stannated macroaggregated albumin (Sn-MAA);
ii) lyophilizing stannated macroaggregated albumin by
   iia) diluting stannated macroaggregated albumin (Sn-MAA) with water and adding human serum albumin;
   iib) lyophilizing the reaction mixture of step iia); and
iii) reconstituting the lyophilized serum with aqueous ^{99m}Tc pertechnetate solution;
wherein steps ia) to ig) are carried out as automated continuous manufacturing process.

In accordance with one embodiment of the present invention, the aforementioned process does not comprise initial purification of the albumin solution used as the starting material.

In accordance with another embodiment of the present invention, the aforementioned process does not comprise separation of the stannous incorporated macroaggregated albumin particles and passing the same through a sizing screen.

In accordance with another embodiment, in the aforementioned process the albumin concentration at step ia) is 0.5% w/v to 3.0% w/v.

In accordance with another embodiment, in the aforementioned process the filtrate at step id) is a colloidal solution with an average particle size between 10 nm-100 nm.

In yet another embodiment, the present invention provides a sterile, non-pyrogenic, single dose, lyophilised pharmaceutical composition, comprising:
i) about 0.02 mg macroaggregated albumin;
ii) not less than 5 μg of stannous chloride;
iii) about 1.0 mg human serum albumin; and
iv) about 0.09-0.15 mg sodium chloride;
wherein the macroaggregated albumin is stannous incorporated macroaggregated albumin and wherein the average particle size of the macroaggregated albumin particles is 25 μm to 35 μm, 95% of the macroaggregated albumin particles are between 10 μm to 70 μm, less than 3% of the particles are below 10 μm and none of the particles are above 100 μm, when measured by optical microscopy and/or automated microscopy and image analysis;
wherein the composition comprises 300,000 to 800,000 macroaggregated albumin particles; and wherein the total tin content of the composition is not more than 13 μg;
wherein the pH of the composition when reconstituted with a 0.9% w/v sodium chloride solution is in the range of 5.0 to 7.0; and
wherein the bacterial endotoxin content is less than 20 EU/vial; and
wherein the composition is contained in a vial comprising an oxygen content of less than 5 percent in the head space.

In accordance with another embodiment, a lyophilized single dose macroaggregated albumin kit, preferably radiolabeled with Technetium Tc-^{99m} will offer strategic and technical advantages over the currently available multiple dose products such as:
i) It will direct benefit to improve the image quality by providing increased assurance and control over the number of particles used in a patient administration;
ii) It will increase the availability of MAA for use in after hours (e.g. night shifts) and weekends that will be flexible to the clinic in terms of time and cost;
iii) It will provide a ready-to-use product within Nuclear Medicine Departments in hospitals;
iv) It will improve the standardization of the injection volume; and
v) It will support in avoiding undesirable sterility issues of multi-dose vials.

In accordance with another embodiment, there is provided a radiopharmaceutical composition comprising a pharmaceutical multiple dose kit composition of macroaggregated albumin for injection combined with a ^{99m}Tc pertechnetate solution.

In accordance with another embodiment, the radiopharmaceutical composition comprises a level of radioactivity in the range of 0.185 MBq to 11100 MBq.

In accordance with another embodiment, there is provided a lyophilized pharmaceutical composition of macroaggregated albumin wherein when the composition is reconstituted with a ^{99m}Tc-pertechnetate solution, the composition has a radiochemical purity between 90% and 110%, preferably at least 105%, 100%, 99%, 98%, 97%, 96%, 95%, 94%, 93%, 92%, 91% at least 12 hours at temperature of 2° C. to 8° C.

In accordance with another embodiment, the present invention also relates to a process of manufacturing aggregated protein-based particles with size ranging from 5 nm to 100 μm.

In accordance with another embodiment, the stannated macroaggregated albumin has a particle density of about 5 million to about 6 million particles per vial after stannous addition with not more than 3% soluble and dispersed human serum albumin impurities content.

In accordance with another embodiment, there is provided a radiopharmaceutical composition of macroaggregated albumin, wherein the radiopharmaceutical composition is used for lung imaging, evaluation of pulmonary perfusion and peritoneovenous (LeVeen) shunt patency.

In accordance with another embodiment there is provided a radiopharmaceutical composition of macroaggregated albumin, wherein the volume of a unit dose is from about 0.1 mL to about 3 mL.

In accordance with another embodiment there is provided a radiopharmaceutical composition of macroaggregated albumin, wherein the radiopharmaceutical composition is used for the diagnosis and/or treatment of ovarian cancer, breast cancer, liver cancer, prostate cancer, thyroid cancer, neuroendocrine tumors (NETs), pancreatic cancer, non-small cell lung cancer (NSCLC), and/or combinations thereof.

In accordance with another embodiment, the single dose kit composition for producing an injectable radiolabeled dispersion of macroaggregated albumin (MAA) particles comprises a non-adsorbing glass vial containing a lyophilized powder comprising macroaggregated albumin, human serum albumin, stannous chloride, tin chloride (stannous and stannic chloride) and sodium chloride. The particle density of the lyophilized powder is in the range of 300,000 to 700,000 and at least 95% or more of particles are between 10 μm to 70 μm in size.

Some radiopharmaceutical compositions are known to have pH drifts of up to 2 pH units due to the presence of radionuclides that highly affect the stability and desired radioactivity of the composition; thus a radiopharmaceutical composition must be pH stable and not significantly drift in pH over time.

In accordance with another embodiment, the lyophilised pharmaceutical composition is pH drift stable, wherein the pH of composition does not drift more than 0.5 during storage. In accordance with another embodiment, the reconstituted radiopharmaceutical composition is pH drift stable, wherein the pH of the reconstituted composition does not change more than 0.5 during storage, preferably does not change more than 0.2 during storage.

Albumin has four partially selective metal binding sites with well-defined metal preferences. Albumin is known for its binding of different metal ions such as copper, zinc, gold, nickel, cadmium, vanadium and platinum. Metal radioisotopes can bind to the metal binding sites present in albumin or alternatively can bind through a chelating agent conjugated to albumin.

The lyophilized macroaggregates can be radiolabeled with a diagnostic or therapeutic radioisotope selected from the group consisting of copper-64, copper-67, gallium-68, gallium-67, gallium-66, lutetium-177, yttrium-86, yttrium-90, technetium-^{99m}, indium-114, indium-111, scandium-47, scandium-44, scandium-43, zirconium-89, bismuth-213, bismuth-212, actinium-225, astatine-211, lead-212, lead-214, gold-198, gold-199, rhenium-188, rhenium-186, and holmium-166, by a process comprising suspending the macroaggregates in a suitable buffer solution, centrifuging the solution, removing the supernatant and resuspending the macroaggregates in a suitable buffer solution followed by incubation with the desired radioisotope solution.

In an exemplary embodiment, radiolabeling of the MAA particles with gallium-68 can be carried out by adding a solution of [68Ga]GaCl₃, obtained from a germanium-68/gallium-68 generator, to a buffered composition of MAA at pH 4 to 5 and heating at a temperature of 40° C. to 95° C.

In another exemplary embodiment, radiolabeling of the MAA particles with fluorine-18 can be carried out by coupling of MAA with N-succinimidyl 4-(18) F-fluorobenzoate at 45° C. in a phosphate buffer of pH 8.5.

In another exemplary embodiment, radiolabeling of the MAA particles with bismuth-212 can be carried out by adding a neutralized solution of bismuth-212 to MAA particles in phosphate buffered saline at pH 7.0 and incubating for 10 minutes at 70° C.

In yet another embodiment, the stable radiolabeled complexes of MAA with lutetium-177 (177Lu) can be obtained by using various bi-functional chelating agents such as DTPA, 1,4,7,10-tetraazacyclododecan-N,N′,N″,N′″-tetraacetic acid (DOTA), and 5p-C-NETA.

In yet another embodiment, the conjugation of MAA with actinium-225 (225Ac) performed by adding the solution of the desired radioisotope to a buffered solution of MAA, optionally in the presence of a suitable chelating agent such as DOTA, DTPA, EDTA, Macropa, Crown and H4py4pa.

In yet another embodiment, the radiolabeling process of conjugating MAA with copper radioisotopes such as copper-61 (61Cu), copper-64 (64Cu), copper-67 (67Cu) can be similarly performed by adding the solution of the desired radioisotope to a buffered solution of MAA in the presence of a suitable chelating agent such as DOTA, DTPA, EDTA, Macropa, Crown, TCMC and H4py4pa.

In yet another embodiment, the conjugation of MAA with lead radioisotopes, 203Pb and 212Pb is performed by adding the solution of the desired radioisotope to a buffered solution of MAA, optionally in the presence of a suitable chelating agent. The preferably used chelating agent is selected from DOTA, TCMC, DOTA-1Py, DOTA-2Py, and DOTA-3Py.

The lyophilized macroaggregates can be reconstituted with a sterile, pyrogen-free solution of sodium pertechnetate ^{99m}Tc forming a suspension of ^{99m}Tc radiolabeled macroaggregates of human serum albumin. For radiolabeling, the source of technetium should be water soluble, preferably in the form of sodium pertechnetate from a ^{99m}Tc generator. The eluate obtained from the ^{99m}Tc generator is added in sufficient quantity to the lyophilized powder in the single dose kit to obtain a standardized suspension (e.g. 1 mL) with a particle count of 400,000-700,000 for use in a single patient.

The radiolabeled pharmaceutical composition of the present invention can be administered and transported using shielded methods known in the art or by technologies disclosed in Jubilant Draximage owned patents U.S. Pat. Nos. 11,554,216, 11,179,518 and 12,102,789.

### EXAMPLES

### Example 1. Preparation and Evaluation of Stannous Macroaggregated Albumin (Sn-MAA)

### Step 1. Preparation of Nanoaggregated Albumin (Nano-HSA):

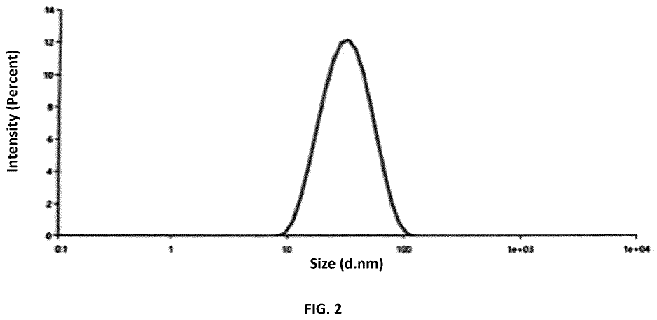

In an automated reactor, human serum albumin (HSA) is diluted with 0.9% NaCl solution in a first vessel under clean room condition. The temperature of the resulting solution is raised to about 75° C. with continuous stirring. NaOH solution is added to raise the pH to 10 and stirring is continued for about 15 to 20 minute at a stirring rate of 200-500 rpm. The reaction mixture is subsequently cooled at a stirring rate of 200 to 400 rpm. The pH of the denatured and nanoaggregated HSA solution thus obtained is adjusted to pH 2.0 to 5.0 by addition of HCl solution. The nanoaggregated albumin solution obtained after this step is translucent to turbid with no visible particles and has an average particle size between 25 nm to 35 nm as measured by dynamic light scattering. The particle size distribution of the nanoaggregates is shown in FIG. 2.

### Step 2. Preparation of Macroaggregated Albumin (MAA):

The nanoaggregated colloidal albumin dispersion obtained in step 1 is sterile filtered and transferred to a sterile second reactor vessel in aseptic area. The pH of the filtrate is adjusted to an isoelectric point of albumin by addition of NaOH at a temperature of 60-70° C. with continuous stirring. The reaction mixture is then cooled to room temperature for formation and precipitation of the macroaggregated albumin particles.

### Step 3. Preparation of Stannous Macroaggregated Albumin (Sn-MAA):

A sterile solution of stannous chloride solution is added to the macroaggregated albumin suspension with stirring under a nitrogen gas environment. The pH of the reaction is adjusted to 4.0 to 6.0 using NaOH solution and heated followed by cooling to room temperature with stirring to obtain stannous macroaggregated albumin (Sn-MAA).

Three batches, namely Example 1.1, Example 1.2 and Example 1.3 of Stannated-macroaggregated albumin (Sn-MAA) are prepared by the process given above. The Sn-MAA obtained from the three batches are technically evaluated for particle size distribution, particle density and presence of soluble and dispersed impurities.

Particle size distribution and particle density was measured using Malvern Morphologi G3 Automated Microscope System are illustrated in Table I below:

TABLE I
Particle size distribution and particle density of Sn-MAA

| | Example | Example | Example |
|---|---|---|---|
| Particle Parameters | 1.1 | 1.2 | 1.3 |
| Particle density/vial | 5,800,000 | 5,900,000 | 5,200,000 |
| Particles larger than 100 μm | None | None | None |
| Particles smaller than 10 μm | 1.2% | 2.9% | 2.6% |

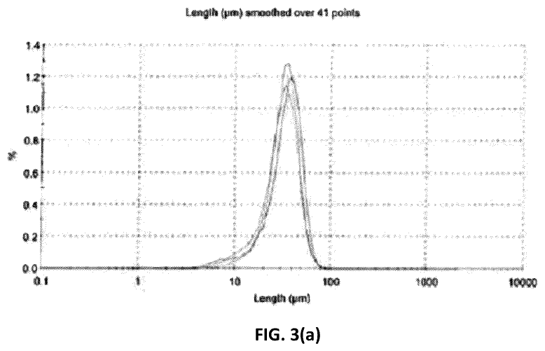

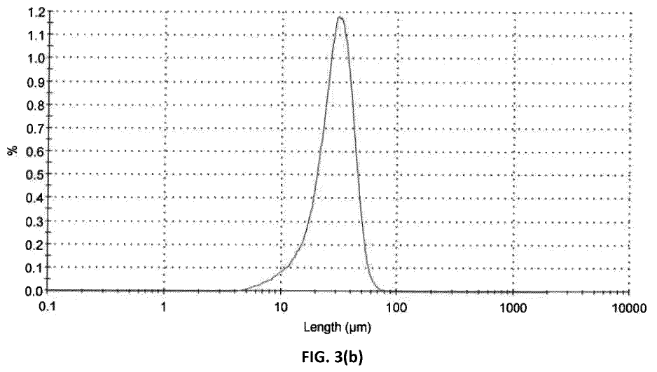

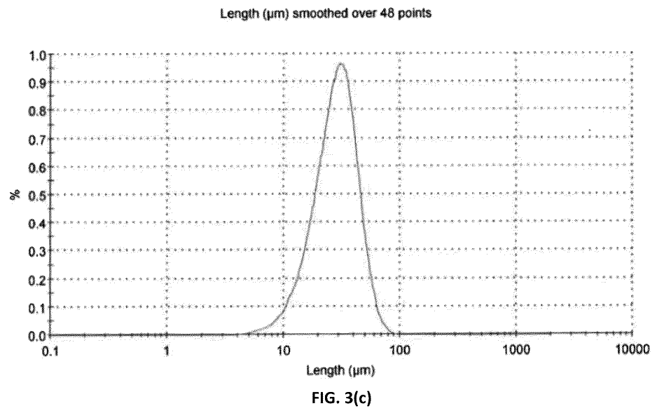

The Sn-MAA prepared by the present invention exhibit low inter-batch variability for particle density and particle size distribution, as shown in FIG. 3(a). The average particle size (length) was between 20 μm to 35 μm (preferably 25 to 35 μm), as shown in FIG. 3(b) and FIG. 3(c).

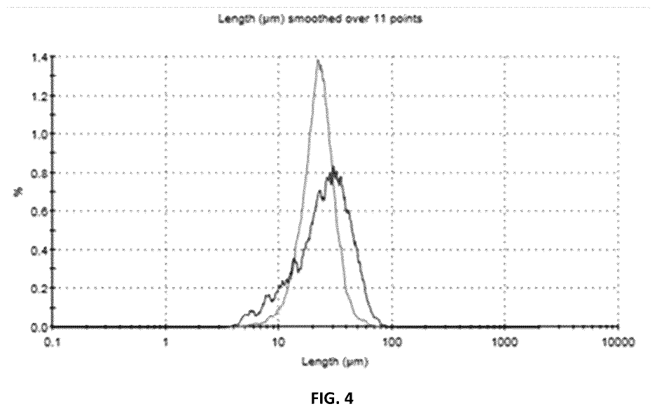

The Sn-MAA lots prepared using the automated reactor also show much lower variability in particle size distribution as compared to the conventional process as given in FIG. 4. The conventional process is a manual process, which requires operator intervention at each step, making the process highly variable.

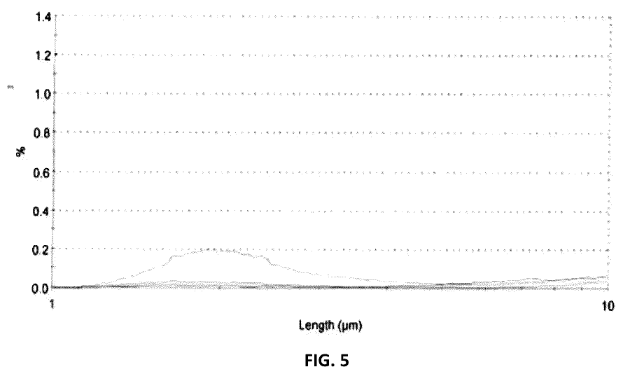

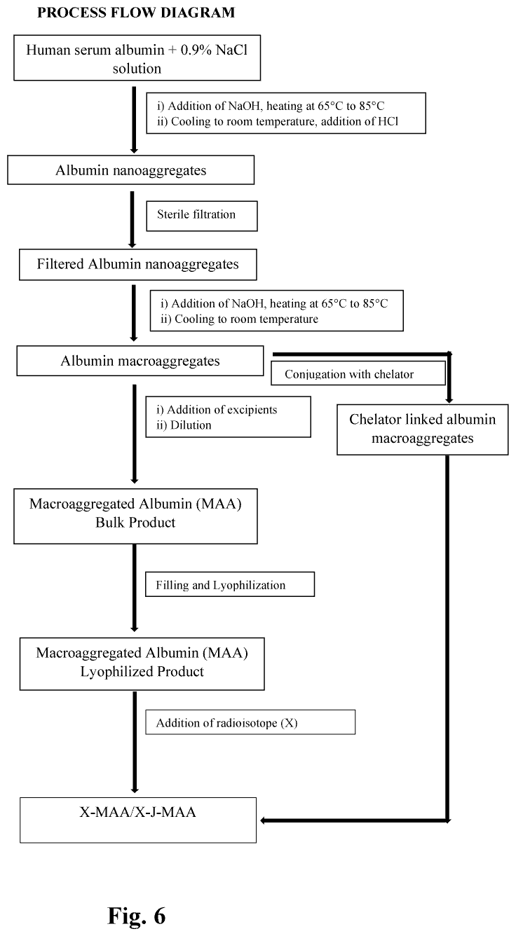

The soluble and dispersed HSA present as impurities in the Sn-MAA bulk are: Sn-HSA microaggregates, Sn-HSA nanoaggregates and Sn-HSA (native protein). Using the Malvern G3 Automatized System to determine the presence of Sn-HSA microaggregates (particles with size between 1 μm to 3 μm) showed negligible presence of the above impurities as given in FIG. 5.

### Example 2: Preparation of MAA Bulk Drug Product for Single Dose Vial (MAA-SDV)

The MAA-SDV drug product is prepared in small-scale (1 L of MAA-SDV bulk) by using three lots of Sn-MAA bulk prepared by the process of Example 1. Dilution of the Sn-MAA bulk with degassed WFI is performed after HSA excipient addition. The dilution factor is calculated according to the targeted particle density of 400,000 to 600,000 particles/vial.

Three batches of MAA Bulk Drug Product for Single Dose (MAA-SDV) are prepared namely, Example 3.1, Example 3.2 and Example 3.3, and their particle size distribution and particle density are measured using Malvern Morphologi G3 Automatized System Analysis as illustrated in Table II below:

TABLE II
Particle size and particle distribution
for bulk drug product (MAA-SDV)

| | Example | Example | Example |
|---|---|---|---|
| Particle Parameters | 3.1 | 3.2 | 3.2 |
| Particle density/vial | 500,280 | 431,160 | 400,600 |
| Particles larger than 100 μm | None | None | None |
| Particles smaller than 10 μm | 2.1% | 2.9% | 2.7% |

### Example 3. Formulation of Macroaggregated Albumin Single Dose Vial (MAA-SDV)

The macroaggregated albumin single dose vial is formulated using the quantity of macroaggregates and adjuncts used per vial for a single dose vial composition as shown in Table III:

TABLE III
Macroaggregate and adjuncts for Single Dose Vial (mg/vial)

| Ingredients | Function | Quantity |
|---|---|---|
| Macroaggregated albumin | API | 0.14 mg-0.26 mg |
| Human serum albumin | Bulking agent | 0.5 mg-5 mg |
| Stannous Chloride | Reducing agent | 0.005 mg |
| Tin Chloride (stannous/stannic) dihydrate | Reducing agent | 0.013 mg |
| Sodium chloride | Isotonicity adjuster | 0.09 mg-0.13 mg |
| Nitrogen gas | Stabilizer | Headspace fill |

The present invention use hydrochloric acid and sodium hydroxide for pH adjustment

### Example 4. Lyophilization of MAA Bulk Drug Product for Single Dose Vial (MAA-SDV) and Evaluation of Impact of Amount of HSA

The MAA-SDV bulk (0.5 ml) is transferred into 10 mL type 1 plus glass vial and is lyophilized. Four MAA-SDV batches are prepared with varied amounts of albumin to evaluate the impact of amount of albumin on the lyophilized plug formation and determine the content of SnCl₂·2H₂O.

The lyophilized composition per vial for the four batches are illustrated in Table IV.

TABLE IV
Composition of MAA-SDV Lyophilization Batches

| Composition per vial | Example 4.1 | | Example 4.2 | | Example 4.3 | | Example 4.4 | |
|---|---|---|---|---|---|---|---|---|
| Macroaggregated albumin | 0.2 | mg | 0.2 | mg | 0.2 | mg | 0.2 | mg |
| Human Serum Albumin (HSA) | 1 | mg | 1 | mg | 0.5 | mg | 5 | mg |
| Tin Chloride (stannous/stannic) | 10 | μg | 10 | μg | 10 | μg | 10 | μg |
| Sodium chloride | 0.1 | mg | 0.1 | mg | 0.1 | mg | 0.1 | mg |

In the study of impact of amount of albumin, it is found that the lyophilization plug deteriorates when the amount of HSA is lower than 1 mg. The batches prepared with 1 mg and 5 mg of HSA have a stable lyophilization plug. However, when the quantity of HSA is 5 mg, it interfered with the analytical method for determination of tin content. Therefore, another 4 batches of lyophilized MAA-SDV product, namely Example 5.1, Example 5.2, Example 5.3 and Example 5.4 are prepared with albumin content of 1 mg/vial and are further evaluated. The lyophilized product is obtained as white plug or powder, free from any visible contamination and are easily re-suspendable on addition of water. The results of the evaluation of lyophilized compositions are illustrated in Table V.

TABLE V
Evaluation of Lyophilized MAA-SDV Product

| | | Example | Example | Example | Example |
|---|---|---|---|---|---|
| Test Description | Acceptance Criteria | 5.1 | 5.2 | 5.3 | 5.4 |
| pH | 5.0-7.0 | Conforms | Conforms | Conforms | Conforms |
| Particle density/vial | 200,000 to 700,000 | 700,000 | 500,000 | 300,000 | 300,000 |
| Particle Size | Not less than 95% ≥10 μm-≤70 μm | 99.1% | 98.1% | 97.1% | 97.0% |
| | Not more than 3% <10 μm | 0.9% | 1.9% | 2.9% | 3.0% |
| | None >100 μm | None | None | None | None |
| Total Tin Assay | Not more than 13 | 11 | 12 | 12 | 12 |
| (μg/vial) | | | | | |
| SnCl₂•₂H₂O Assay | Not less than 5 | Conforms | Conforms | Conforms | Conforms |
| (μg/vial) | | | | | |
| Macroaggregated | 0.14-0.26 | Conforms | Conforms | Conforms | Conforms |
| albumin (mg/vial) | | | | | |
| Human serum | 0.7-1.3 | 0.9 | 1.0 | 0.9 | 1.0 |
| albumin (mg/vial) | | | | | |
| Sodium chloride | 0.09-0.13 | 0.11 | 0.13 | 0.12 | 0.13 |
| (mg/vial) | | | | | |
| Bacterial endotoxin | Not more than 20 EU/Vial | Conforms | Conforms | Conforms | Conforms |

The symbols used herein refer following : “≥” greater than or equal to; “≤” less than or equal to; “<” less than and “>” greater than.

### Example 5. Radiolabeling of Lyophilized MAA Single Dose Vial (MAA-SDV)

Example 4 formulation vial is placed in a suitable lead pot and stored under a mild atmosphere of nitrogen. A sterile venting needle is inserted through the rubber septum. Up to 88800 MBq of technetium ^{99m}Tc generator eluate diluted with about 15.0 mL Sodium Chloride Injection is injected into the vial. Before removing the syringe from the vial, 2 mL of gas is withdrawn from above the solution and then the venting needle is removed. The contents of the vial are-mixed gently to ensure complete solubilization of the powder and incubated at room temperature for 15 minutes. The reconstituted product is stored at 2° C.-8° C.

Three batches, Example 5.1, Example 5.2 and Example 5.3, of reconstituted product are prepared by the process given above and are tested for radiochemical purity, centrifugation test and bio-distribution studies. Initial tests are done at post-radiolabeling at 15-30 minutes and Delayed tests are done at 12 hours post-radiolabeling. The tests for radiochemical purity (% of the total radioactivity found as aggregated albumin) and centrifugation test (% of the radioactivity found as dispersed radioactivity) are performed as per United States Pharmacopoeia monograph on Technetium Tc^{99m} Albumin Aggregated Injection USPNF_M80590. Further, bio-distribution tests are performed by approximately 1 hour post-reconstitution of the vial using Technetium Tc^{99m} solution, the test animal is injected intravenously between 0.074 MBq and 0.740 MBq of injection, in a volume not exceeding 0.2 mL. The animal is sacrificed 5 to 10 minutes after the injection and liver, kidneys and lungs of each animal are carefully removed by dissection. The organs are placed in suitable counting containers, and the radioactivity is determined in counts per minute, in each container with a Wizard 2480 Gamma counter. The animal testing studies as per the invention were performed according to the animal ethical guidelines. The results are illustrated in Table VI.

TABLE VI
Evaluation of MAA-SDV Products Reconstituted with Tc-^{99m} Sodium Pertechnetate Solution

| Test | Acceptance | Example 5.1 | | Example 5.2 | | Example 5.3 | |
|---|---|---|---|---|---|---|---|
| Description | Criteria | Initial | Delayed | Initial | Delayed | Initial | Delayed |
| Radiochemical | Not less than | 100% | 100% | 100% | 100% | 100% | 100% |
| Purity | 90% | | | | | | |
| Centrifugation | Not more than | Conforms | Conforms | Conforms | Conforms | Conforms | Conforms |
| Test | 10% | | | | | | |
| Bio-distribution | Lungs: Not | 87% | 85% | 92% | 95% | 97% | 91% |
| | less than 80% | | | | | | |
| | Liver: Not | 1% | 1% | None | None | 1% | 1% |
| | more than 5% | | | | | | |
| | Kidneys: Not | 1% | 2% | 1% | 1% | 1% | 2% |
| | more than 5% | | | | | | |

### Example 6. Preparation of Drug Product for Multiple Dose Vial (MAA-MDV)

The MAA-MDV drug product is prepared in small-scale (1 L of MAA-MDV bulk) by using three lots of Sn-MAA bulk prepared by the process of Example 1. Dilution of the Sn-MAA bulk with degassed water for injection is done after the HSA excipient is added. The dilution factor is calculated according to the targeted particle density of 300,000 to 700,000 particles/vial. Then 0.5 ml of the MAA-MDV bulk is transferred into 10 mL type one plus glass vials and lyophilized.

### Example 7. Formulation of Macroaggregated Albumin Multiple Dose Vial (MAA-MDV)

The quantities of macroaggregates and adjuncts used per vial for a multiple dose vial composition are illustrated in Table VII.

TABLE VII
Macroaggregates and adjuncts per vial

| Ingredients | Function | mg/vial |
|---|---|---|
| Macroaggregated albumin | API | 2.2 mg-3.0 mg |
| Human serum albumin | Bulking agent | 3.5 mg-6.5 mg |
| Stannous Chloride | Reducing agent | 0.005 mg |
| Tin Chloride (stannous | Reducing agent | ≥0.06 mg |
| and stannic), dihydrate | | |
| Sodium chloride | Isotonicity adjuster | 0.96 mg-1.44 mg |
| Nitrogen gas | Stabilizer | Headspace fill |

In one aspect, the present invention use hydrochloric acid and sodium hydroxide for pH adjustment

### Example 8. Preparation of Lyophilized MAA-MDV Product

The lyophilized product is obtained as a white plug or powder, free from any visible contamination and is easily re-suspendable on addition of water. The lyophilized products are evaluated for pH, loss on drying, particle size, particle density, and other tests. The results are illustrated in Table VIII.

TABLE VIII
Results of Lyophilized Product MAA Multiple Dose Vial

| | | Example | Example | Example | Example |
|---|---|---|---|---|---|
| Test Description | Acceptance Criteria | 8.1 | 8.2 | 8.3 | 8.4 |
| pH | 5.0 to 7.0 | Conforms | Conforms | Conforms | Conforms |
| Loss on drying | Not more than 5% | <1% | 1% | <1% | <1% |
| Particle density/vial | 3,000,000 to 7,000,000 | 3,000,000 | 3,000,000 | 3,000,000 | 3,000,000 |
| Particle Size | Not less than 95% ≥10 μm-≤70 μm | 96% | 97% | 96% | 97% |
| | Not more than 3% <10 μm | 2.5% | 2.8% | 2% | 2% |
| | None >100 μm | None | None | None | None |
| SnCl₂•₂H₂O Assay | Not less than 0.06 | Conforms | Conforms | Conforms | Conforms |
| (μg/vial) | | | | | |
| Macroaggregated | 2.2-3.0 | Conforms | Conforms | Conforms | Conforms |
| albumin (mg/vial) | | | | | |
| Human serum | 3.5-6.5 | 5.6 | 5.8 | 6.0 | 5.8 |
| albumin (mg/vial) | | | | | |
| Sodium chloride | 0.96-1.44 | 1.29 | 1.27 | 1.36 | 1.27 |
| (mg/vial) | | | | | |
| Bacterial endotoxin | Not more than 20 EU/Vial | Conforms | Conforms | Conforms | Conforms |

### Example 9. Radiolabeling of Lyophilized MAA Multiple Dose Vial (MAA-MDV)

Example 8 formulation vial is placed in a suitable lead pot and stored under a mild atmosphere of nitrogen. A sterile venting needle is inserted through the rubber septum. Up to 88800 MBq of technetium ^{99m}Tc generator eluate diluted with about 15.0 mL Sodium Chloride Injection is injected into the vial. Before removing the syringe from the vial, 2 mL of gas is withdrawn from above the solution and then the venting needle is removed. The contents of the vial are mixed gently to ensure complete solubilization of the powder and further incubated at room temperature for 15 minutes. The reconstituted product is stored at 2-8° C. for 12 hrs.

Three batches of reconstituted product Example 9.1, Example 9.2 and Example 9.3 are prepared by process given above and are tested for radiochemical purity, centrifugation test and bio-distribution studies. Initial tests are done at post-radiolabeling at 15-30 minutes and Delayed tests are done at 12 hours post-radiolabeling. The tests for radiochemical purity and centrifugation test is performed as per United States Pharmacopoeia monograph on Technetium Tc^{99m} Albumin Aggregated Injection USPNF_M80590. Bio-distribution tests are determined by approximately 1 hour post-reconstitution of the vial using Technetium Tc^{99m} solution, the animal is injected intravenously between 0.074 MBq and 0.740 MBq of injection, in a volume not exceeding 0.2 mL. The animal is sacrificed 5 to 10 minutes after the injection and liver, kidneys and lungs of each are carefully removed by dissection. The organs are placed in suitable counting containers, and the radioactivity is determined in counts per minute, in each container with a Wizard 2480 Gamma counter. The results are illustrated in Table IX.

TABLE IX
Results of Radiolabeled Product MAA Multiple Dose Vial

| Test | | Example 9.1 | | Example 9.2 | | Example 9.3 | |
|---|---|---|---|---|---|---|---|
| Description | Radioactivity | Initial | Delayed | Initial | Delayed | Initial | Delayed |
| Radiochemical | Not less than | 100% | 100% | 100% | 100% | 100% | 100% |
| purity | 90% | | | | | | |
| Centrifugation | Not more than | Conforms | Conforms | Conforms | Conforms | Conforms | Conforms |
| Test | 10% | | | | | | |
| Bio-distribution | Lungs: Not | 87% | 85% | 92% | 95% | 97% | 91% |
| | less than 80% | | | | | | |
| | Liver: Not | 1% | 1% | None | None | 1% | 1% |
| | more than 5% | | | | | | |
| | Kidneys: Not | 1% | 2% | 1% | 1% | 1% | 2% |
| | more than 5% | | | | | | |

The single dose and multiple dose compositions of the present invention are evaluated for their storage stability under ambient and accelerated storage conditions as per International Council for Harmonisation of Technical Requirements of Pharmaceuticals for Human Use (ICH) guidelines. The prepared dosage forms exhibited desirable technical attributes like pH, particle size distribution and particle density, the studies showed no variation in the physical appearance and re-suspendability. Initial and delayed radiochemical purity of all the stability samples are found to be 100% and conformed to the desirable limit. Based on the stability results a shelf-life of 2 years can be anticipated for both the single dose and multiple dose lyophilized compositions.

The results presented in the examples demonstrate absence of lot-to-lot variation and excellent uniformity of particle size distribution and particle density parameters between batches proving that rigorous process design and control of process parameters can avoid lot-to-lot variation and can result in macroaggregated albumin products with excellent storage stability suitable for precise dose delivery.

### Example 10. Preparation of X-J-MAA

Example 10.1. Preparation of radiolabeled conjugate compound, where chelating agent J is not required for radiolabeling: The lyophilized macroaggregated albumin or stannated macroaggregated albumin prepared according to the present invention is radiolabeled with a radionuclide X at a suitable temperature and suitable pH in the range of about 3.0 to about 9.0, wherein X can be 68Ga, 18F, 212Bi and the like.

Example 10.2. Preparation of radiolabeled conjugate compound by radiolabeling with chelating agent J: The lyophilized macroaggregated albumin or stannated macroaggregated albumin prepared according to the present invention is reacted with a chelating agent J to obtain J-MAA. The J-MAA is then radiolabeled with a radionuclide X at a suitable temperature and suitable pH in the range of about 3.0 to about 9.0, wherein X can be 177Lu, 203Pb, 212Pb, 61Cu, 64Cu, 225Ac and the like.

What is claimed:

1. A process for preparing a reconstituted radiopharmaceutical composition comprising radiolabelled macroaggregated albumin particles, the process comprising the steps of:
i) preparing stannous macroaggregated albumin (Sn-MAA) by:
ia) mixing human serum albumin with saline solution;
ib) heating the albumin solution and adjusting the pH to between 8.0 to 12.0 with stirring at a rate of 200 to 500 rpm;
ic) cooling to 20 to 25° C. with stirring at a rate of 200 to 400 rpm;
id) adjusting the pH to 2.0 to 5.0 and sterile filtering the solution to obtain a filtrate;
ie) adjusting the pH of the filtrate to 4.0 to 6.0;
if) heating the filtrate at 50 to 80° C. with stirring at a rate of 200 to 400 rpm and cooling to obtain macroaggregated albumin; and
ig) reacting the macroaggregated albumin with stannous chloride to obtain stannous macroaggregated albumin (Sn-MAA);
ii) lyophilizing stannous macroaggregated albumin by:
iia) diluting the stannous macroaggregated albumin (Sn-MAA) with water and adding human serum albumin;
iib) lyophilizing the reaction mixture of step iia); and
iii) reconstituting the lyophilized composition with an aqueous 99mTc pertechnetate solution;
wherein steps ia) to ig) are carried out as an automated continuous process using a reactor equipped with computerized process control and steps id) to iib) are carried out in an aseptic environment.

2. The process of claim 1, wherein the process does not comprise initial purification of the albumin solution used as starting material.

3. The process of claim 1, wherein the process does not comprise separation of the stannous incorporated macroaggregated albumin particles and passing the same through a sizing screen.

4. The process of claim 1, wherein the albumin concentration at step ia) is 0.5% w/v to 3.0% w/v.

5. The process of claim 1, wherein the filtrate at step id) is a colloidal solution with average particle size between 10 nm to 50 nm.

6. The process of claim 1, wherein the average particle size of the macroaggregated albumin particles is 25-35 μm and the average number of macroaggregated albumin particles in a single dose is about 500,000 and the maximum difference in the number of macroaggregated albumin particles between two single doses of the lyophilized pharmaceutical composition is about 400,000 particles.

7. The process of claim 1, wherein 95% of the macroaggregated albumin particles are between 10-70 μm, less than 3% of the particles are below 10 μm and none of the particles are above 100 μm, when measured by optical microscopy and/or automated microscopy and image analysis.

8. The process of claim 1, wherein the pH is adjusted by addition of an acid and/or a base.

9. The process of claim 8, wherein the acid is hydrochloric acid and the base is sodium hydroxide.

10. A process for preparing a single dose or a multiple dose lyophilized composition comprising macroaggregated albumin particles, the process comprising the steps of:
i) preparing stannous macroaggregated albumin (Sn-MAA) by:
ia) mixing human serum albumin with saline solution to form an albumin solution;
ib) heating the albumin solution and adjusting the pH to between 8.0 to 12.0 with stirring at a rate of 200 to 500 rpm;
ic) cooling the albumin solution to 20 to 25° C. with stirring at a rate of 200 to 400 rpm;
id) adjusting the pH of the albumin solution to 2.0 to 5.0 and sterile filtering the albumin solution to obtain a filtrate;
ie) adjusting the pH of the filtrate to 4.0 to 6.0;
if) heating the filtrate at 50 to 80° C. with stirring at a rate of 200 to 400 rpm and cooling to obtain macroaggregated albumin; and
ig) adding a sterile solution of stannous chloride to the macroaggregated albumin suspension with stirring under nitrogen gas environment to form a reaction mixture; and
ih) adjusting the pH of the reaction mixture to 4.0 to 6.0 and heating followed by cooling to room temperature with stirring to obtain stannous macroaggregated albumin (Sn-MAA); and
ii) lyophilizing the stannous macroaggregated albumin by:
iia) diluting the stannous macroaggregated albumin (Sn-MAA) with water and adding human serum albumin; and
iib) transferring aliquots of the mixture of diluted Sn-MAA and human serum albumin into sterile glass vials, lyophilizing the contents of the sterile glass vials and sealing the sterile glass vials under a nitrogen atmosphere;
wherein steps ia) to ih) are carried out as an automated continuous process using a reactor.

11. The process of claim 10, wherein the process does not comprise an initial step of purification of the albumin solution used as starting material.

12. The process of claim 10, wherein the process does not comprise separation of the stannous incorporated macroaggregated albumin particles and passing the stannous incorporated macroaggregated albumin particles through a sizing screen.

13. The process of claim 10, wherein the albumin concentration at step ia) is 0.5% w/v to 3.0% w/v.

14. The process of claim 10, wherein the filtrate at step id) is a colloidal solution with an average particle size between 10 nm to 50 nm.

15. The process of claim 10, wherein the average particle size of the macroaggregated albumin particles is 25-35 μm and the average number of macroaggregated albumin particles in a single dose is about 500,000 and the maximum difference in the number of macroaggregated albumin particles between two single doses of the lyophilized pharmaceutical composition is about 400,000 particles.

16. The process of claim 10, wherein 95% of the macroaggregated albumin particles are between 10-70 μm, less than 3% of the particles are below 10 μm and none of the particles are above 100 μm, when measured by optical microscopy and/or automated microscopy and image analysis.

17. The process of claim 10, wherein the pH is adjusted by addition of an acid and/or a base.

18. The process of claim 17, wherein the acid is hydrochloric acid.

19. The process of claim 17, wherein the base is sodium hydroxide.

20. The process of claim 10, further comprising reconstituting the lyophilized composition by mixing the lyophilized composition with an aqueous 99mTc pertechnetate solution.
